(19) United States
(12) Patent Application Publication
(10) Pub. No.: US 2012/0283833 A1
(43) Pub. Date: Nov. 8, 2012

(54) ARTICULAR CARTILAGE TREATMENT METHOD

(76) Inventor: JAMES K. BRANNON, LEAWOOD, KS (US)

(21) Appl. No.: 12/961,491

(22) Filed: Dec. 6, 2010

Related U.S. Application Data
(60) Provisional application No. 61/266,900, filed on Dec. 4, 2009.

(51) Int. Cl. A61F 2/28

(52) U.S. Cl. 623/16.11

(57) ABSTRACT

The present invention provides an method to alleviate a deteriorated condition at an articular joint site and for repair of damaged cartilage the method including the steps of imaging for the presence of osteonecrosis in the underlying bone, forming a cylindrical plug comprising articular cartilage and osteonecrotic bone using an endoscopic trephine, inspecting the removed cylindrical plug and the joint site for additional articular cartilage and osteonecrotic bone, selectively debriding osteonecrotic bone material associated with the bone cavity to achieve desired vascular characteristics for receiving a prepared bone graft material within the bone cavity, and overlying the received bone graft material with cartilage tissue at the cartilage tissue receiving surface.

### CROSS-REFERENCE TO RELATED APPLICATION

This application claims the benefit of the prior filed U.S. provisional application No. 61/266,900 filed Dec. 4, 2009 and prior filed U.S. nonprovisional application Ser. No. 12/820,133 filed Jun. 21, 2010 which are incorporated herein by reference.

### FIELD OF THE INVENTION

The present invention is broadly concerned improvements in the treatment of articular cartilage damage, injury, or lesions and, more particularly, to a treatment method which includes treatment of bone conditions underlying the cartilage damage.

### BACKGROUND OF THE INVENTION

Articular cartilage on the surface of bones in joints, most particularly the knee, ankle and hip joints, is susceptible to deterioration caused by injury, disease, and aging. Untreated articular cartilage lesions have a limited ability to heal and may promote degenerative changes in the joint. Damage to the structure and function of the articular cartilage leads to pain, loss of range of motion, crepitation, swelling, and eventually deformity. Surgical efforts to restore articular cartilage involve a wide variety of surgical procedures.

Prosthetic devices have been used to replace damaged or destroyed articular cartilage. Although there are several prosthetic devices which can be used in the replacement of damaged or destroyed articular cartilage, prosthetic devices have several disadvantages. For example, cements which are used to attach prosthetic devices to bones may loosen and eventually fail. In addition, fragmented cement can move into the joints and associated lymph tissue and cause inflammation and further damage. Further, cements may result in the formation of fibrous tissue between the bone and the prosthesis. Another major disadvantage associated with the use of prosthesis is that the prosthetic device may be larger than the damaged cartilage that needs to be replaced, thereby requiring removal of portions of healthy bone and/or cartilage in order to accommodate the prosthetic device. Hence, the need remains for a system for repairing and regenerating articular cartilage which avoids the problems associated with prosthetic devices.

Surgical efforts to restore articular cartilage involve a wide variety of surgical procedures. These include debridement procedures, such as chondral shaving; marrow stimulation procedures such as abrasion arthroplasty; penetration of the subchondral bone by drilling or microfracture; cartilage resurfacing and regrowth procedures, such as autologous osteo-chondral transplantation; and the use of artificial matrix, periosteum transplantation, and autologous chondrocyte transplantation. None of these procedures has shown consistent formation of normal articular cartilage and none has been indicated for arthritic knees. To restore lost function, alleviate pain, and prevent degenerative changes within the knee, the ideal cartilage treatment should be minimally invasive and result in hyaline cartilage regrowth in the area of the defect, fully integrated with native bone and surrounding cartilage. Few surgical options are indicated for large lesions, patients with severe arthrosis, or for older patients.

A technique which has been applied to articular cartilage repair is articular cartilage paste grafting. One paste grafting technique for the knee joint used lavage, debridement, and subchondral fracture to stimulate autologous, mesenchymal stem cell proliferation, differentiation, and growth factor release. To present a three-dimensional autogenous cartilage matrix with chondrocytes to large defects, an osteocartilaginous paste graph was harvested from the interchondylar notch, crushed into a paste, and impacted into the fractured chondral defect. The combined morselized paste of articular cartilage and subchondral bone is hypothesized to augment the mesenchymal stem cell supply from vascularized subchondral marrow access, and may present the necessary cellular signals and conductive matrix to produce an appropriate repair tissue. Animal studies suggested the superiority of paste grafting to controls and histologic regeneration of cartilage repair surfaces in defects both in arthritic knees and acute trauma. The technical feasibility of the placement and persistence of the osteocartilaginous paste has been established by both animal and human clinical studies. Further information about this type of articular cartilage paste grafting can be found in: Articular Cartilage Paste Grafting to Full-Thickness Articular Cartilage Knee Joint Lesions: A 2- to 12-Year Follow-up by Kevin R. Stone, M.D. et al., from Arthroscopy: The Journal of Arthroscopic and Related Surgery, volume 22, No. 3 (March) 2006: pages 291-299, which is incorporated herein by reference.

Although articular cartilage paste grafting has succeeded with some patients, success has been limited, has not been universal, and in others relapses have occurred. Thus, there remains a need for alternative remedies in the treatment of underlying bone conditions associated with articular cartilage degeneration.

### SUMMARY OF THE INVENTION

It has been found that in some patients articular cartilage degeneration is not a primary condition but a symptom of localized deterioration of the underlying bone. The present invention provides embodiments of a method to diagnose and treat an underlying articular bone condition to more successfully treat articular cartilage degeneration.

Bones are typically composed of a hard outer tissue referred to as cortical bone tissue, a spongy inner tissue referred to as cancellous bone tissue, and other types of tissues. At the various joints within the body, the ends of the bones are covered with cartilage which acts as connective tissue and also as padding. In healthy bone condition, the cortical and cancellous bone tissues cooperate to provide the necessary strength to support strenuous activities as well as resilience to absorb impacts. However, certain conditions can cause deterioration of bone tissue known as osteonecrosis. Osteonecrosis changes the character of the bone tissue resulting in a loss of resilience. Over time, the hardened bone tissue at areas of engagement with other bones increases compressive stress on the articular cartilage lining the bones of the joint, thereby causing the cartilage to deteriorate.

There are around 6.5 million fractures per year in the United States, of which approximately 15% are difficult to heal. For those fractures in which the healing is slow (delayed union) or does not occur (nonunion), there are few effective therapies at present. The most common method of treatment is insertion of bone from the individual (autologous) or from alternate sources (autogenous) into the defect. In this procedure, bone is removed from a variety of sources, most commonly the pelvis following a surgical incision made in the hip area. The donor bone tissue is inserted at the nonhealing fracture site, and additional support may be provided by an orthopedic rod or plate. More than 250,000 bone grafts are performed annually in the United States in an attempt to assist the body in regenerating new bone lost by trauma or disease. Poor fracture healing is associated with chronic pain and prolonged ambulatory impairment and must often be treated by surgical intervention. This has considerable economic implications for healthcare providers. External fixation devices may stabilize fractures at risk from poor healing, although a lack of viable bone at the fracture site may result, at best, in the production of unstable bone that is prone to refracture. Although bone grafting is generally successful, it suffers from the limited amount of donor tissue that may be available, and the patient may suffer side effects such as numbness or tingling at the donor site, infection, or prolonged pain. An alternative therapy involves the use of pulsed electromagnetic fields, which have been shown to have effects on many aspects of bone formation and healing. This includes the induction of endothelial and bone cell proliferation, the formation of capillary sprouts, the stimulation of matrix formation, and calcification.

In an embodiment of the present invention, the method of articular cartilage treatment includes an initial diagnosis of the condition of the bone underlying damaged cartilage. This can be determined by radiant imaging such as magnetic resonance imaging (MRI), computed tomography (CT), or the like. The images obtained are analyzed to determine the presence and dimensional extent of articular osteonecrosis. If osteonecrosis is found, then treatment of the underlying bone along with the damaged cartilage is indicated.

In an embodiment of the present invention, generally the affected joint is entered arthroscopically, if possible, and the damaged articular cartilage is debrided, followed by debriding of the desired bone tissue, i.e. necrotic. The removed bone tissue is replaced with bone graft material. Then the removed articular cartilage is replaced by cartilage regrowth material, such as by an articular cartilage paste graft. In an embodiment of the present invention, the cartilage and necrotic bone tissue can be debrided at the same time using a trephine instrument of a diameter comparable to the extent of the damaged cartilage and necrotic bone tissue. Such a trephine instrument is tubular and has a sharpened circular distal end to form a circular cutting or coring edge. Alternatively, the distal end can be provided with saw teeth.

With the coring edge, the surgical site is entered and the coring edge is placed perpendicularly to the bone surface. A sharp blow on a proximal end of the trephine drives the coring edge into the bone, capturing a cylindrical plug consisting of the damaged cartilage and necrotic bone tissue. Alternatively, a saw tooth tipped trephine can be rotated, as by a small motor, and engaged with the cartilage and bone to remove the diseased portions. The site is inspected to determine if additional debriding is required and, if so, areas of necrotic bone and damaged cartilage may be debrided.

When debriding is complete, the bone cavity is filled with a bone graft or regrowth material to restore the bone. Various types of bone graft materials are in common use.

Malleable putty is sometimes used to correct bone defects that may be caused by trauma, pathological disease, surgical intervention or other situations where defects need to be managed in osseous surgery. It is important to have the defect filler in the form of a stable, viscous putty to facilitate the placement of the bone growth medium into the surgical site which is usually uneven in shape and depth. The surgeon will take the putty on a spatula or other instrument and trowel it into the site or take it in his/her fingers to shape the bone inducing material into the proper configuration to fit the site being corrected.

Many products exist to treat this surgical need. One example is autologous bone particles or segments recovered from the patient. When removed from the patient, it is wet and viscous from the associated blood. This works very well to heal the defect but requires significant secondary surgery resulting in lengthening the surgery, extending the time the patient is under anesthesia and increasing the cost. In addition, a significant increase in patient morbidity is attendant in this technique as the surgeon must take bone from a non-involved site in the patient to recover sufficient healthy bone, marrow and blood to perform the defect filling surgery. This leads to significant post-operative pain.

Another product group involves the use of inorganic materials to provide a matrix for new bone to grow at the surgical site. These inorganic materials include hydroxyapatite obtained from sea coral or derived synthetically. Either form may be mixed with the patient's blood and/or bone marrow to form a gel or a putty. Calcium sulfate or plaster of Paris may be mixed with water to similarly form a putty. These inorganic materials are osteoconductive but are bioinert and do not absorb or become remodeled into natural bone. They consequently remain in place indefinitely as a brittle, foreign body in the patient's tissue.

Allograft bone is a logical substitute for autologous bone. It is readily available and precludes the surgical complications and patient morbidity associated with autologous bone as noted above. Allograft bone is essentially a collagen fiber reinforced hydroxyapatite matrix containing active bone morphogenic proteins (BMP) and can be provided in a sterile form. The demineralized form of allograft bone is naturally both osteoinductive and osteoconductive. The demineralized allograft bone tissue is fully incorporated in the patient's tissue by a well established biological mechanism. It has been used for many years in bone surgery to fill the osseous defects previously discussed.

It is well known in the art that for several decades surgeons have used a patient's own blood as a vehicle in which to mix the patient's bone chips or bone powder, or demineralized bone powder so as to form a defect filling paste. Blood is a useful carrier because it is available from the bleeding operative site, is non-immunogenic to the patient and contains bone morphogenic proteins which facilitate wound healing through new bone growth. However, stored blood from other patients has the deficiencies that any blood transfusion would have such as blood type compatibility, possibility of transmission of disease and unknown concentration of BMP which are to a great extent dependent upon the age of the donor.

When the bone graft material has been placed in the bone cavity, a cartilage regrowth material is applied to the surface of the restored bone. Application of the cartilage regrowth material may include the articular cartilage past grafting described above.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a diagrammatic fragmentary cross section of an area of an articular bone and illustrates damaged articular cartilage and necrosis of the underlying bone tissue.

FIG. 2 is a view similar to FIG. 1 and illustrates debridement of the damaged articular cartilage and the necrotic bone tissue.

FIG. 3 is a view similar to FIG. 2 and illustrates replacement of the necrotic bone tissue with a bone graft material and replacement of the damaged articular cartilage with articular cartilage paste graft material.

FIG. 4 is a flow diagram illustrating the principal steps of an embodiment of the articular cartilage treatment method according to the present invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

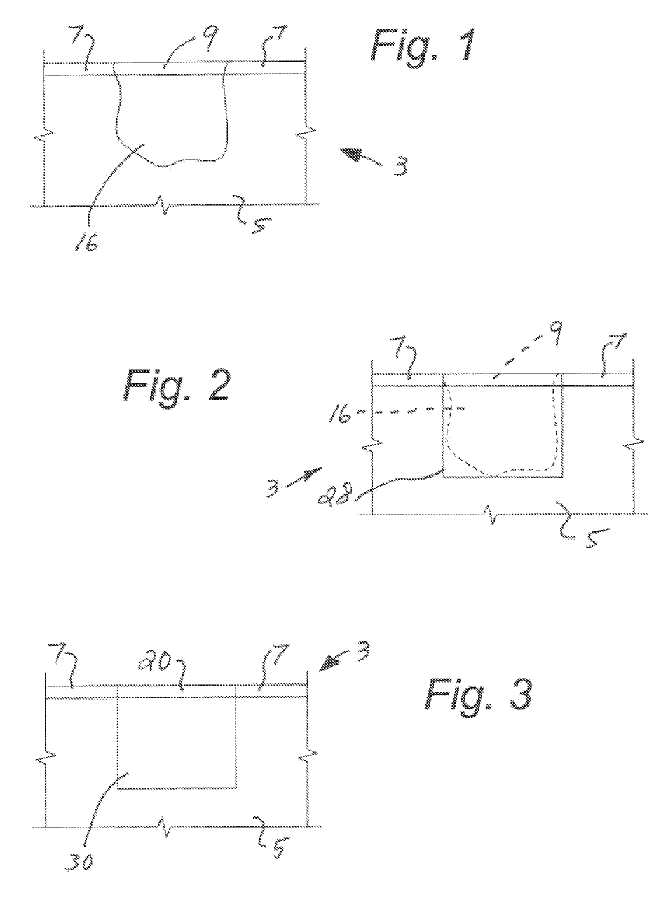

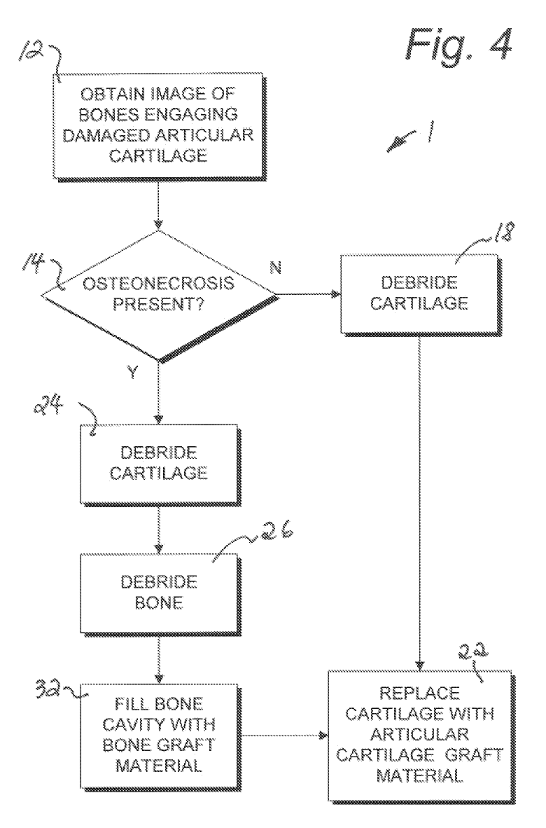

This application incorporates by reference applicant's prior pending U.S. patent application 61/218,757, attached hereto along with the referenced article by Kevin R. Stone. Referring to the drawings in more detail, the reference numeral 1 (FIG. 4) generally designates an embodiment of an articular cartilage treatment method according to the present invention. The method 1 is practiced to alleviate a deteriorated condition at an articular or bone joint site 3 (FIGS. 1-3). At the joint site 3, an articular bone segment 5 is illustrated with a layer of articular cartilage 7 positioned on an external surface thereof. Symptoms of articular cartilage damage 9 are present. Preferably, the joint site 3 is entered arthroscopically to minimize the extent of incisions and to promote faster recovery of the patient.

According to an embodiment of the treatment method 1, one or more images of the articular site 3 are obtained at step 12 (FIG. 4), as by magnetic resonance imaging. Analysis of the images is made at step 14 to diagnose the possible presence of osteonecrosis 16 underlying the portion of damaged cartilage 9. If osteonecrosis 16 is not diagnosed, the joint site 3 is entered arthroscopically using an endoscopic trephine instrument (not shown), advanced proximally towards the bone joint site optionally with use of a drill and fluoroscopy. In one embodiment the trephine instrument may include a sharpened distal end, a proximal end, and an elongated longitudinal surface circumferentially extending thereabout. Next the damaged articular cartilage 9 is debrided at step 18, using for example, the sharpened distal end. The debrided cartilage material is removed from the bone joint site with one end of the trephine instrument in communication with at least one of a suction source and an irrigation source. After removal of the debrided material, an articular cartilage graft material 20 may be transported to the bone joint site through the trephine instrument at step 22 and implated at the area of debridement. The articular cartilage graft step 22 may be carried out in a manner similar to that described in the Stone et al. paper referred to previously.

If osteonecrosis 16 is determined to be present in the underlying bone 5, the cartilage is debrided at step 24, and the osteonecrotic bone tissue 16 is debrided at step 26. The debriding steps 24 and 26 can be carried out separately using appropriate instruments. Alternatively, most of the debriding steps 24 and 26 can be accomplished simultaneously at the bone joint site, using the endoscopic trephine instrument having an appropriate diameter, as described previously. A trephine instrument similar to that disclosed in U.S. Pat. No. 6,007,496, which is incorporated herein by reference, could be employed in the combined debriding steps 24 and 26. Combined debriding of the damaged cartilage 9 and necrotic bone tissue 16, using the endoscopic trephine instrument, a cylindrical plug sample 28 is selected (FIG. 2) of cartilage 7 and bone 5. The plug sample 28 may then be removed and examined, and remaining cartilage 7 and bone 16 may be endoscopically inspected using a visual instrument extended through the endoscopic trephine instrument to determine if any remaining areas of diseased tissues are present. If so, additional debriding 24 and 26 can take place.

Additional debriding may be utilized to achieve desired vascular conditions. During bone and/or cartilage debridement, the damaged tissue acquires a “wounded phenotype.” Generally, tissue repair results from a number of temporally coordinated processes driven by locally released mediators. The first event is immediate and consists of the activation of the coagulation cascade and the formation of a blood clot. Shortly afterward there follows an acute inflammatory response resulting in tissue edema and cytokine and growth factor release. Then follows the first stage of collagen repair, involving deposition and the formation of granulation tissue, which becomes a new and temporary weak tissue. The third and final process is the second phase of collagen repair, resulting in extracellular matrix remodeling, angiogenesis, and the reproduction of full-strength tissue. During a normal healing process naturally occurring growth factors and cytokines are produced. In addition to their role in blood clot formation, platelets and mesenchymal cells generate a number of growth factors that are found in wound fluid, including TGF-, platelet-derived growth factor (PDGF), epidermal growth factor, vascular endothelial growth factor, TGF-β, and insulin-like growth factor-I (IGF-I). In this acute inflammatory response, neutrophil migration is induced by PDGF, interleukin-1 and -8, tumor necrosis factor (TNF)-, granulocyte macrophage-colony stimulating factor, and granulocyte-colony stimulating factor.

The release of these growth factors assist in the repair of damaged bone tissue, which include acidic fibroblast growth factor (aFGF), basic fibroblast growth factor (bFGF), PDGF, and TGF-β. Thus multiple growth factors and cytokines assist in bone tissue repair. The BMPs that are produced by osteoblasts and mesenchymal cells merely represent one level of tissue specificity among a wide variety of stimulatory proteins. Thus the wounded phenotype “marks” injured tissue separately from uninjured healthy tissue, thereby enhancing the healing process of the healthy tissue or slowing the spread of a variety of diseased tissue such as diabetic retinopathy dermal scarring in soft tissue and the production of extraneous bone.

Upon completion of cartilage and bone debriding 24 and 26, the bone cavity is filled with a bone graft material 30 at step 32. The bone growth material may be comprosed of bone-inducing agents, such as demineralized bone powder, calcium phosphate, hydroxyapatite, organoapatite, titanium oxide, poly-L-lactic and polyglycolic acid or copolymer thereof, alone or in combination and in amounts corresponding to the surrounding bone characteristics.

The regrowth material may be optionally dispensed using a growth agent delivery system, an endoscopic passageway extending towards the bone joint site and an inflatable orthopedic device adapted for receiving the growth agent as disclosed in U.S. application Ser. No. 12/820,133 which is incorporated by reference. In such a growth agent delivery system, the inflatable orthopedic device includes an outer membrane and a seal for selectively receiving growth agent. The endoscopic passageway includes an endoscopic connector adapted to receive materials and communicate the material through its channel. The endoscopic passageway is further adapted for fluid communication from the growth agent delivery system to the inflatable orthopedic device. The inflatable orthopedic device is adapted to dispense bone growth which may include bone marrow cells to the debrided bone tissue. In another embodiment a stabilizer may be utilized in association with the growth agent delivery system, the stabilizer being adapted for securing the inflatable orthopedic device in receipt of the growth agent within the bone cavity. The received growth agent within the debrided bone presents an osseous surface consistent with viable bone tissue and adapted for receiving replaced cartilage thereby stimulating cartilage repair. Afterwards, the bone growth agent is positioned within the bone cavity, the removed cartilage 9 is replaced with the articular cartilage graft material 20 at step 22. The bone joint site 3 is then exited and the entry incision is sutured. In this manner the use of the inflatable growth agent delivery system allows for treatment of posterior femoral or posterior tibial lesions.

Treatment of the osteonecrosis 16 regenerates healthy bone tissue which is more resilient than the osteonecrotic tissue, such that when the cartilage graft 20 heals, there is less chance of recurrence of the cartilage damage. Additional information regarding the treatment of osteonecrosis can be found in U.S. Pat. No. 7,445,595, which is incorporated herein by reference.

It is to be understood that while certain forms of the present invention have been illustrated and described herein, it is not to be limited to the specific forms or arrangement of parts described and shown.

The inventor hereby states his intent to rely on the Doctrine of Equivalents to determine and assess the reasonably fair scope of the present invention as pertains to any apparatus not materially departing from but outside the literal scope of the invention as set forth in the following claims.

Having thus described the invention, what is claimed as new and desired to be secured by Letters Patent is as follows:

1. A method to alleviate a deteriorated condition at an articular joint site and for repair of damaged cartilage said method comprising the steps of:
imaging the articular site for the presence of osteonecrosis in the underlying bone;
forming a cylindrical plug comprising articular cartilage and osteonecrotic bone using an endoscopic trephine having a sharpened distal end, said cylindrical plug corresponding to a bone cavity and a cartilage receiving surface, said cartilage receiving surface overlying said bone cavity;
inspecting the removed cylindrical plug and the joint site for additional articular cartilage and osteonecrotic bone;
selectively debriding osteonecrotic bone material associated with the bone cavity to achieve desired vascular characteristics for receiving a prepared bone graft material within the bone cavity, and
overlying said received bone graft material with cartilage tissue at said cartilage tissue receiving surface whereby said bone graft material presents an osseous surface adapted for promoting said cartilage tissue.

2. The method of claim 1 further comprising the steps of:
inserting an endoscope having an endoscopic passageway between an incision and the bone joint site;
positioning an inflatable orthopedic device through said endoscopic passage towards said surgical site, said inflatable orthopedic device adapted for receipt of bone growth agent; and
dispensing said growth agent to said bone joint site from said inflatable orthopedic device.
